(19) United States
(12) Patent Application Publication
(10) Pub. No.: US 2011/0313172 A1
(43) Pub. Date: Dec. 22, 2011

(54) PROCESS FOR THE PREPARATION OF 1-[5-(3-CHLORO-PHENYL)-ISOOXAZOL-3-YL]-ETHANONE AND (R)-1-[5-(3-CHLORO-PHENYL)-ISOOXAZOL-3-YL]-ETHANOL

(75) Inventors: Hans Åström, Sodertalje (SE); Elfyn Jones, Sodertalje (SE)

(73) Assignee: ASTRAZENECA AB, Sodertalje (SE)

(21) Appl. No.: 13/140,565

(22) PCT Filed: Dec. 11, 2009
(86) PCT No.: PCT/SE09/51405

§ 371 Date: Sep. 1, 2011

Related U.S. Application Data
(60) Provisional application No. 61/138,741, filed on Dec. 18, 2008.

(51) Int. Cl. C07D 261/08

(52) U.S. Cl. 548/248; 548/247

(57) ABSTRACT

The present invention provides a process for the preparation of the compound 1-[5-(3-chloro-phenyl)-isooxazol-3-yl]-ethanone: Wherein the compound 5-(3-chlorophenyl)-isooxazol-3-carboxylate is reacted with CH3MgX. The present invention also provides a process for the preparation of (R)-1-[5-(3-chloro-phenyl)-isooxazol-3-yl]-ethanol: Wherein 1-[5-(3-chloro-phenyl)-isooxazol-3-yl]-ethanone is reduced to (R)-1-[5-(3-chloro-phenyl)-isooxazol-3-yl]-ethanol.

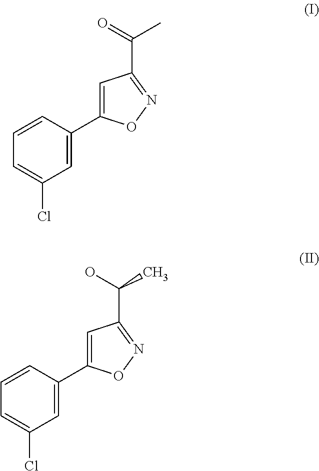

### FIELD OF THE INVENTION

The present invention relates to a new process for large-scale production of 1-[5-(3-chloro-phenyl)-isoxazole-3-yl]-ethanone and, optionally, (R)-1-[5-(3-chloro-phenyl)-isoxazole-3-yl]-ethanol. These compounds are useful as intermediates for manufacturing pharmaceutically active larger compounds.

### TECHNICAL BACKGROUND

4-(5-{(1R)-1-[5-(3-chlorophenyl)isoxazol-3-yl]ethoxy}-4-methyl-4H-1,2,4-triazol-3-yl)pyridine is an antagonist of the mGluR5 receptor. Accordingly, this compound is expected to be well suited for treatment of mGluR5-mediated disorders, such as acute and chronic neurological and psychiatric disorders, gastrointestinal disorders and chronic and acute pain disorders. This and similar compounds are disclosed in WO, A1, 2007/040982. This patent application also describes a process where (R)-1-[5-(3-chloro-phenyl)-isoxazole-3-yl]-ethanol, an intermediate compound in the synthesis of 4-(5-{(1R)-1-[5-(3-chlorophenyl)isoxazol-3-yl]ethoxy}-4-methyl-4H-1,2,4-triazol-3-yl)pyridine, is manufactured in an eight-step process.

The process of WO, A1, 2007/040982 is a multi-step process that is suitable for laboratory scale. Accordingly, there is a need for an improved process, which is possible to carry out in larger scale, and which ideally is simple, cost effective, and without harmful impact on the environment.

### SUMMARY OF THE INVENTION

The present invention provides a process for the preparation of the compound 1-[5-(3-chloro-phenyl)-isoxazol-3-yl]-ethanone of the formula

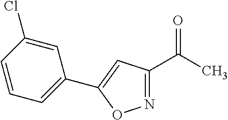

wherein
the compound ethyl 5-(3-chlorophenyl)-isoxazole-3-carboxylate of the formula

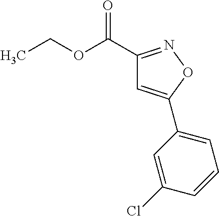

dissolved in a solvent, is reacted with CH₃MgX dissolved in a solvent, wherein X is chlorine or bromine, thereby providing the compound 1-[5-(3-chloro-phenyl)-isoxazol-3-yl]-ethanone dissolved in said solvent.

As disclosed herein, the term “C₁₋₁₂alkyl” relates to a linear or branched alkyl group comprising 1-12 carbon atoms, such as but not limited to, methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl, n-heptyl, n-octyl, n-nonyl, n-decyl, n-undecyl and n-dodecyl.

Preferably, the solvent is selected from the group of aromatic hydrocarbons, such as toluene, and ortho-, meta- and para-xylene, as well as ethers such as 2-methyl tetrahydrofuran, tetrahydrofuran, diethyl ether, tert-butyl methyl ether or mixtures thereof.

The reagent CH3MgX, may be charged to the reaction as a solution in a solvent such as toluene, tetrahydrofuran, 2-methyl tetrahydrofuran or mixtures thereof.

Preferably, the reaction between said ethyl 5-(3-chlorophenyl)-isoxazole-3-carboxylate and said CH₃MgX is carried out in the presence of a tertiary amine, such as triethyl amine. Also other tertiary aliphatic amines, linear or branched, such as tri-N-butylamine or N-alkylpiperidines, may be considered.

It is preferred that the reaction mixture and surplus of said CH₃MgX is quenched by adding an acid aqueous solution, such as 6 M HCl.

It is further preferred that the organic reaction mixture after removal of said acid aqueous mixture is treated with an aqueous base such as sodium hydroxide.

In a preferred embodiment, the present invention also provides a process for preparing (R)-1-[5-(3-chloro-phenyl)-isoxazol-3-yl]-ethanol.

In a first preferred alternative, this compound may be prepared by a process comprising the steps of:

i) carrying out the above disclosed process for preparing 1-[5-(3-chloro-phenyl)-isoxazol-3-yl]-ethanone;

ii) providing (S)-2-methyl-CBS (Corey, Bakshi, Shibta) oxaborolidine and borane or a borane complex, dissolved in a solvent;

iii) adding 1-[5-(3-chloro-phenyl)-isoxazol-3-yl]-ethanone dissolved in a solvent to the solution obtained in step ii); and

iv) recovering (R)-1-[5-(3-chloro-phenyl)-isoxazol-3-yl]-ethanol from the reaction.

In one embodiment of the invention, 1-[5-(3-chloro-phenyl)-isoxazol-3-yl]-ethanone dissolved in said second solvent, is added to the solution obtained in step ii), during a time period of up to 4 h.

Preferably, the borane in step ii) is borane dimethyl sulfide. Alternative borane sources such as borane tetrahydrofuran, borane triethylamine and borane N,N-diethylaniline complexes may be used in the process. Preferably, the solvent is tetrahydrofuran, 2-methyl tetrahydrofuran or toluene.

It is preferred that an excess of borane is quenched by adding an alcohol, such as methanol, after completion of formation of (R)-1-[5-(3-chloro-phenyl)-isoxazol-3-yl]-ethanol.

Preferably, (R)-1-[5-(3-chloro-phenyl)-isoxazol-3-yl]-ethanol is recovered by crystallization. A suitable solvent or solvent mixture for the crystallization of (R)-1-[5-(3-chloro-phenyl)-isoxazol-3-yl]-ethanol may be selected from the group of aromatic hydrocarbons such as toluene and xylenes, ethers such as 2-methyl tetrahydrofuran, tetrahydrofuran, diethyl ether and tert-butyl methyl ether, alkanes such as n-heptane and cyclohexan, polar aprotic solvents such as dimethylsulfoxide, dimethylformamide as single crystallization solvent or in any combination with or without water present.

In a second alternative, (R)-1-[5-(3-chloro-phenyl)-isoxazol-3-yl]-ethanol may be prepared by an enzymatic process involving the use of a stereospecific alcohol dehydrogenase capable of catalyzing formation of (R)-1-[5-(3-chloro-phenyl)-isoxazol-3-yl]-ethanol together with a suitable co-factor selected from the group of NADH and NADPH, comprising the steps of:

1) carrying out the above disclosed process for preparing 1-[5-(3-chloro-phenyl)-isoxazol-3-yl]-ethanone;

2) adding said 1-[5-(3-chloro-phenyl)-isoxazol-3-yl]-ethanone to a suitable reaction medium containing a sufficient amount of said alcohol dehydrogenase together with said co-factor; and

3) recovering (R)-1-[5-(3-chloro-phenyl)-isoxazol-3-yl]-ethanol from said suitable reaction medium.

A suitable reaction medium for the reaction may be a buffered aqueous solution containing an alcohol such as 2-propanol. Said buffered aqueous solution may be a triethanolamine buffer having a pH within the range of 7.0-8.5. Examples of suitable alcohol dehydrogenases include IEP Ox29 and IEP Ox58, which are manufactured by IEP GmbH, DE and obtainable from DSM Pharmaceutical Products, Geleen, NL. Preferably, said co-factor is NADH. (R)-1-[5-(3-chloro-phenyl)-isoxazol-3-yl]-ethanol may be recovered from the reaction medium by extraction with ethyl acetate, recovering the organic phase and evaporating the solvent. Alternatively, (R)-1-[5-(3-chloro-phenyl)-isoxazol-3-yl]-ethanol may be recovered from the reaction medium by extraction with methyl tert-butyl ether, recovering the organic phase and crystallizing the product from a mixture of methyl tert-butyl ether and n-heptane.

In a third alternative, (R)-1-[5-(3-chloro-phenyl)-isoxazol-3-yl]-ethanol may be prepared by an asymmetric hydrogenation comprising the steps of:

1) carrying out the above disclosed process for preparing 1-[5-(3-chloro-phenyl)-isoxazol-3-yl]-ethanone;

2) adding said 1-[5-(3-chloro-phenyl)-isoxazol-3-yl]-ethanone to a suitable reaction medium containing a solvent and a catalytic amount of a transition metal based catalyst in the presence of a strong base such as potassium tert-butoxide and applying hydrogen gas at atmospheric or elevated pressure.

3) recovering (R)-1-[5-(3-chloro-phenyl)-isoxazol-3-yl]-ethanol from said suitable reaction medium.

In a fourth alternative, (R)-1-[5-(3-chloro-phenyl)-isoxazol-3-yl]-ethanol may be prepared by an asymmetric transfer hydrogenation comprising the steps of:

1) carrying out the above disclosed process for preparing 1-[5-(3-chloro-phenyl)-isoxazol-3-yl]-ethanone;

2) adding said 1-[5-(3-chloro-phenyl)-isoxazol-3-yl]-ethanone to a suitable reaction medium containing a solvent and a catalytic amount of a transition metal based catalyst such as (R,R)-TsDPEN)(p-cymene)Ru(II)Cl in presence of either
(i) a strong base such as potassium tert-butoxide and 2-propanol; or
(ii) a solution of triethylamine and formic acid; and

3) recovering (R)-1-[5-(3-chloro-phenyl)-isoxazol-3-yl]-ethanol from said suitable reaction medium.

In a fifth alternative, (R)-1-[5-(3-chloro-phenyl)-isoxazol-3-yl]-ethanol may be prepared by a dynamic kinetic resolution comprising the steps of:

1) carrying out the above disclosed process for preparing 1-[5-(3-chloro-phenyl)-isoxazol-3-yl]-ethanone;

2) adding said 1-[5-(3-chloro-phenyl)-isoxazol-3-yl]-ethanone to a reaction mixture containing a solvent and a reducing agent such as sodium borohydride, thus producing 1-[5-(3-chloro-phenyl)-isoxazol-3-yl]-ethanol as a racemic mixture;

3) adding said (rac) 1-[5-(3-chloro-phenyl)-isoxazol-3-yl]-ethanol to a reaction mixture containing an enzyme such as a lipase, a racemerization agent and an acyl donor such as vinyl acetate, thus producing acetic acid (R)-1-[5-(3-chloro-phenyl)-isoxazol-3-yl]-ethyl ester;

4) adding said (R)-1-[5-(3-chloro-phenyl)-isoxazol-3-yl]-ethyl ester to a suitable reaction medium containing a solvent and a base such as lithium hydroxide; and

5) recovering (R)-1-[5-(3-chloro-phenyl)-isoxazol-3-yl]-ethanol from said suitable reaction medium.

### DETAILED DESCRIPTION OF THE INVENTION

As already stated above, one embodiment of the present invention relates to a process for producing 1-[5-(3-chloro-phenyl)-isoxazole-3-yl]-ethanone.

Still an embodiment of the invention is directed to a process for making (R)-1-[5-(3-chloro-phenyl)-isoxazole-3-yl]-ethanol.

The new manufacturing process of the present invention may be described in the following way:

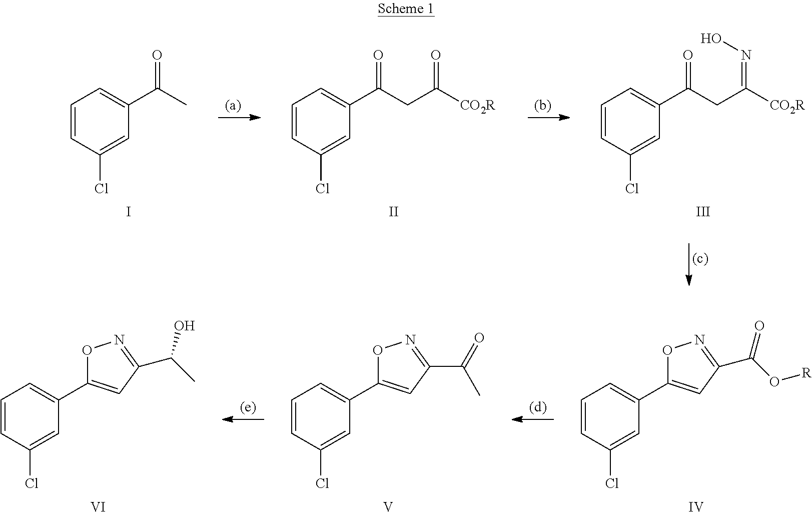

In step a-c of the manufacturing process, a compound of formula IV is prepared.

In step a compound I is reacted with a compound of formula VII

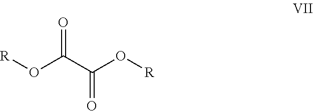

where R is a linear or branched C₁-C₁₂ alkyl;

in the presence of a solvent and a base, particularly an alkoxide base, to (after quench and acidic work-up) give a compound of formula II where R is a linear or branched C₁₋₁₂alkyl;

followed by reacting the compound of formula II, wherein R is defined as above, with hydroxyl amine either as free base or as a salt, in particular hydroxylamine hydrochloride, in a solvent to obtain a compound of formula III which is left in the reaction mixture in the presence of acid, in particular hydrochloric acid, to obtain a compound of formula IV which may be isolated; or

reacting a compound of formula IV, wherein R is defined as above, with a mixture of methyl magnesium bromide and triethylamine in a solvent to (after quench and work-up) give a compound of formula V, which is isolated, or followed by

reacting a compound of formula V with a mixture of borane and (S)-2-Me-CBS oxaborolidine in a solvent to (after quench and work-up) obtain a compound of formula VI that may be isolated.

Alternatively, the compound of formula V may be exposed to an alcohol dehydrogenase together with an appropriate co-factor in a suitable reaction medium in order to produce a compound of formula VI.

Alternatively, the compound of formula V may be exposed to a transition metal based catalyst in presence of a strong base and hydrogen gas to produce a compound of formula VI.

Alternatively, the compound of formula V may be exposed to a transition metal based catalyst in the presence of

(i) a strong base such as potassium tert-butoxide and 2-propanol; or
(ii) a solution of triethylamine and formic acid;
providing a compound of formula VI.

Alternatively, the compound of formula V may be reduced to a racemic mixture of VI by adding a reducing agent such as sodium borohydride to a suitable reaction media followed by enzymatic resolution by a lipase in the presence of an acyl donor, such as vinyl acetate. The resulting ester may be cleaved off using a basic reagent such as lithium hydroxide, providing a compound of formula VI.

The reaction steps a) b) and c) may be performed in a solvent. Suitable solvents are alcohols such as ethanol, methanol and 2-propanol and ethers such as tetrahydrofuran and 2-methyl tetrahydrofuran.

The total amount of solvents used in process steps a-c may vary in the range of from about 2-100 (v/w) volume parts per weight of starting material (compound I), particularly in the range from 6-30 (v/w) volume parts per weight of starting material.

A suitable base may be an alkoxide base such as sodium ethoxide or sodium methoxide. The skilled person will appreciate that a suitable base with respect to the R-group on compound II-IV should be used.

The temperature for step a-c may be in the range of from about 0° C.-100° C., particularly in the range of from 50-80° C.

The temperature for step d) should be in the range of from about −10° C.-50° C., particularly in the range of from −5° C.-20° C.

The temperature for step e) should be in the range of from about −10° C.-50° C.

The invention will now be described with reference to the working examples. These examples are provided for information purposes and are not intended to restrict the scope of the present invention.

### EXPERIMENTAL WORK

In the examples below, a Micromass Q-TOF micro instrument has been used to record mass spectra and NMR spectra were recorded using a Bruker 400 mHz Instrument.

### Example 1

### Preparation of ethyl-4-(3-chlorophenol)-2,4-dioxobutanoate

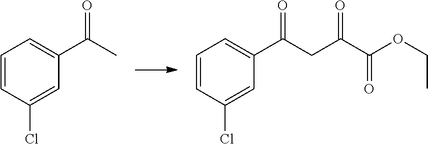

Sodium ethoxide (97.9 g, 1.44 mol) was added in portions to a solution of 3-chloro-acetophenone (178.5 g, 1.15 mol) and diethyl oxalate (195 ml, 1.44 mol) in ethanol (1 l) at 0° C. The mixture was stirred at room temperature for 1 h and was then heated for 2 h at 70° C. After cooling, the reaction was quenched with 1.44 mol HCl in isopropyl alcohol. The resulting mixture was used in subsequent example.

### Example 2

### Preparation of ethyl 5-(3-chlorophenol)-isoxazole-3-carboxylate via 4-(3-chloro-phenyl)-2-[(E)-hydroximino]-4-oxobutyric acid ethyl ester

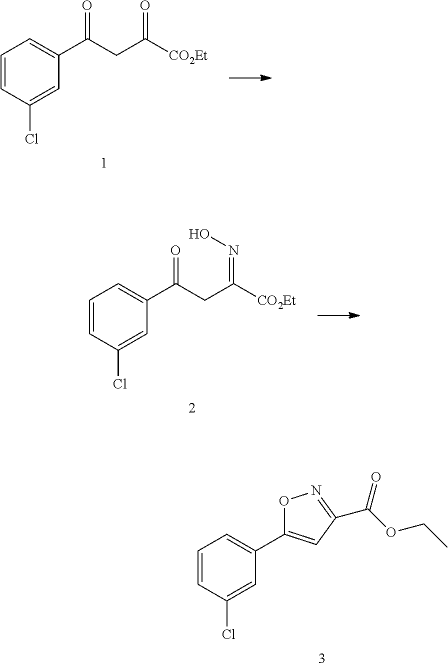

To a solution of ethyl-4-(3-chlorophenyl)-2,4-dioxobutanoate (1) in ethanol is added either hydroxylamine (50% in water) or hydroxylamine hydrochloride. In case the former reagent is used, the reaction halts at the intermediate oxime ester (2). Acid (e.g. hydrochloric acid) is further added to achieve ring closure leading to formation of ethyl 5-(3-chlorophenyl)-isoxazole-3-carboxylate (3). If hydroxylamine hydrochloride is used, ring closure is obtained without further addition of acid.

### Method a, Use of Hydroxylamine (50% in Water)

196 g (0.76 mol) ethyl-4-(3-chlorophenyl)-2,4-dioxobutanoate (1) dissolved in ethanol (960 ml) from previous reaction stage was used. To this solution, hydroxylamine, 50% in water (46.6 ml, 0.76 mol) was added over approximately 1 h at 60° C. After completion of addition, the reaction was kept under stirring for 15 min. Complete conversion had then been obtained. Hydrochloric acid (5 M in propanol, 167.4 ml) was added over 0.5 h, after which the mixture was kept under stirring for 1 h. The temperature was then adjusted to 22° C. and water (384 ml) was added to the reaction mixture over 1 h to crystallize the product. The temperature was then adjusted to and kept at 5° C. for 1 h. Finally, the product was isolated by filtration, washed with (i) 2×360 ml ethanol/water 2:1 and (ii) 360 ml water and dried at 40° C. under reduced pressure. 154.1 g (assay 98.6%) ethyl 5-(3-chlorophenyl)-isoxazole-3-carboxylate corresponding to an isolated yield of 79% was isolated.

MS ESI-TOF analysis in negative mode of intermediate (2) gave [M−H]⁻=268 m/z

### Method b, Use of Hydroxylamine Hydrochloride

196 g (0.76 mol) ethyl-4-(3-chlorophenyl)-2,4-dioxobutanoate (1) dissolved in ethanol (960 ml) from previous reaction stage was used. To this solution, hydroxylamine hydrochloride (55.5 g, 0.8 mol) was added in one portion at 5° C. The reaction temperature was then adjusted and kept at 60° C. for 1 h. Complete conversion had been obtained. The temperature was adjusted to 22° C. and water (384 ml) was added to the reaction mixture over 1 h to crystallize the product. The temperature was then adjusted to and kept at 5° C. for 1 h. Finally, the product was isolated by filtration, washed with (i) 2×360 ml ethanol/water 2:1, and (ii) 360 ml water and dried at 40° C. under reduced pressure. 162.3 g (assay 98.5%) ethyl 5-(3-chlorophenyl)-isoxazole-3-carboxylate corresponding to an isolated yield of 84% was isolated.

### Example 3

### Preparation of 1-[5-(3-chloro-phenyl)-isoxazol-3-yl]-ethanone

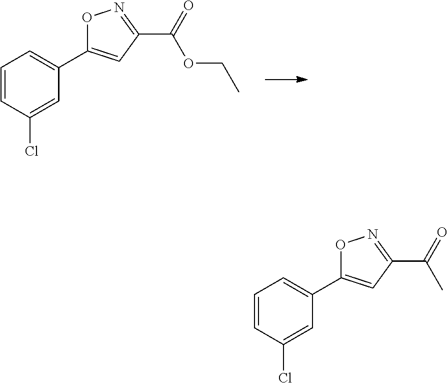

80 g (313.4 mmol) ethyl-5-(3-chlorophenyl)-isoxazole-3-carboxylate was suspended in 360 ml 2-methyl-tetrahydrofuran (Me-THF) in a dry 2 l reactor. The temperature was adjusted to −5° C. A pale slurry was obtained in the reactor.

447.8 ml (626.9 mmol) methyl magnesium bromide (1.4 M solution in toluene-THF) was mixed with 264.8 ml (1880.6 mmol) triethyl amine in a dry dropping funnel. The Grignard solution was then added to the mixture in the reactor over at least 4 h. The dropping funnel was rinsed with 40 ml Me-THF and the wash solution was transferred to the reactor.

459.7 ml 6 M HCl (aq) was carefully added to quench the reaction mixture. The charge was exothermic and evolution of methane gas was noted. After completion of quench, the temperature was adjusted to 50° C. and the water phase was separated off and discarded. The organic phase was washed with 160 ml water. 5.6 g 45% NaOH (aq) was added to the organic phase to convert aldol-condensed by-products formed during quench back to the desired ketone. The mixture was kept under vigorous stirring for 30 min at 50° C. 137.9 ml 0.5 M hydrochloric acid was added at 50° C., to pH<3. The water phase was separated off. Finally, the organic phase was washed with 160 ml water. A yield of 95% was achieved based on assay determination of the solution.

¹H NMR (CDCl₃) 7.82 (m, 1H), 7.70 (m, 1H), 7.47 (m, 2H), 6.93 (s, 1H), 2.72 (s, 3H); High resolution MS Q-TOF analysis in positive mode gave [M+H]⁺=222 m/z; The molecular formula: C₁₁H₉ClNO₂ was confirmed with an accuracy of −0.3 ppm.

### Example 4

### Preparation of (R)-1-[5-(3-Chloro-phenyl)-isoxazol-3-yl]-ethanol (II)

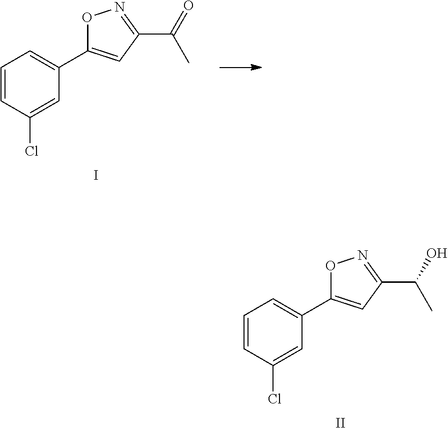

37.0 mL (37.04 mmol) (S)-2-Methyl-CBS-oxaborolidine (1M solution in toluene) and 22.4 mL (222.25 mmol) borane dimethylsulfide were mixed and diluted with 82 mL 2-methyltetrahydrofurane. The resulting solution was heated to 45° C. A solution of 1-[5-(3-Chloro-phenyl)-isoxazol-3-yl]-ethanone (I), 82.1 g (370.4 mmol) dissolved in 410 mL 2-methyltetrahydrofurane and 164 mL toluene (from previous reaction stage) was added to the CBS-borane solution over approximately 4 h. The reaction had reached complete conversion after the addition of the ketone solution. The inner temperature was then set to 0° C. and 103 mL methanol was added to quench excess borane. The quenched reaction mixture was then extracted with (i) 287 mL 2M HCl and (ii) 287 mL water. The organic phase was further evaporated to dryness and the residue was dissolved in 245 mL toluene. The temperature was adjusted to 20° C. after which crystallization was initiated by addition of 0.2 g II (seed crystals). The crystallization mixture was kept for 30 min after which 492 mL n-heptane was added as anti-solvent over 6 h. The crystallization mixture was then chilled from 20 to 0° C. over 6 h. The crystals were then filtered off and washed with (i) 165 mL n-heptane-toluene 2/1 and (ii) 165 mL n-heptane. The crystals were finally dried at 40° C. under reduced pressure. 66.4 g product corresponding to an isolated yield of 80% was isolated. Enantiomeric excess was determined to >98%.

### Example 5

### Enzymatic preparation of (R)-1-[5-(3-Chloro-phenyl)-isoxazol-3-yl]-ethanol (II)

12 g 1-[5-(3-Chloro-phenyl)-isoxazol-3-yl]-ethanone (I) was added to 18 ml 50 mM triethanolamine buffer, pH 8.0, and 36 ml 2-propanol. After adjusting the pH to 8.0 using 1 M NaOH, 6 mg NADH was added. The reaction mixture was kept at 35-40° C. and 5.2 ml of alcohol dehydrogenase preparation IEP Ox29 (manufactured by IEP GmbH, DE, obtainable from DSM Pharmaceutical Products, Geleen, NL) was added to start the reduction. Periodically, samples were taken and analyzed, after filtration over a 45 μm filter, by means of chiral HPLC. After 18 hours of reaction the conversion reached 99.7%.

To 30 g of the enzyme reaction mixture, 25 ml water was added. As a consequence, a part of the product precipitated. Then, 50 ml ethylacetate was added in order to extract the product. Separation of the layers was good. This was followed by two additional extractions using 25 ml ethylacetate. The combined organic layers were filtered over a decalite pre-coated filter. Finally, the solvent was removed on a rotavapor, under reduced pressure, at 45° C. This resulted in 6 g off-white solid.

### Example 6

### Catalytic enantioselective transfer hydrogenation of 1-[5-Chloro-phenyl)-isoxazol-3-yl]-ethanone to give (R)-1-[5-(3-Chloro-phenyl)-isoxazol-3-yl]-ethanol

Under an inert atmosphere 8.3 g (37.5 mmol) 1-[5-Chloro-phenyl)-isoxazol-3-yl]-ethanone is mixed with 23.8 mg (37.4 μmoles) (R,R)-TsDPEN)(p-cymene)Ru(II)Cl. A solution containing 13.8 g (299.6 mmoles, 11.3 mL Formic Acid) and 18.9 g (187.2 mmoles; 26.1 mL) Triethylamine is added. The slurry that was obtained was kept under stirring overnight. The reaction was then sampled showing a virtually complete conversion of starting material to (R)-1-[5-(3-Chloro-phenyl)-isoxazol-3-yl]-ethanol in 95.4% enantio selectivity. The reaction mixture was then diluted with 35 mL toluene and extracted with 2×35 ml, water. The organic layer was further concentrated by evaporation under reduced pressure. The residue was purified by crystallization from a mixture of toluene and n-heptane. Finally, the crystals were isolated by filtration, washed with n-heptane and dried under reduced pressure at 40° C.

Screening experiments have been carried out according to the table below. Selectivity for the S-Isomer of the alcohol is presented in the table. The use of the other isomer of the catalysts will give the desired compound, (R)-1-[5-(3-Chloro-phenyl)-isoxazol-3-yl]-ethanol (R-Isomer).

### Screening Protocol:

To each of 48 2 mL vials was charged:

100 μL Et3N. Then the metal precursors and N-monosulfonylated diamines were added as stock solutions according to the table below to generate 48 combinations (40 μL of 0.008M solution of the metal precursor in DMF, and 55 μL of the N-monosulfonylated diamines 0.013M in iPrOH/toluene 5:3).

The mixtures were agitated at room temp. for 30 minutes to generate the active catalysts. Then 200 μL of the hydride donor (Et₃N/HCOOH 5:8 molar ratio) was added followed by 500 μL of a solution of the ketone in THF (40 mg/mL) to all vials.

The mixtures were then agitated for 2 hours at 25° C. The mixtures were then sampled (20 μL) and diluted with iPrOH 500 μL+heptane 500 μL.

| | | Dichloro- | | Pentamethyl- |
|---|---|---|---|---|
| | Benzene- | di-mu-chlorobis- | Dichloro- | cyclopenta- |
| | ruthenium(II) | (pentamethyl- | (p-cymene)- | dienyliridium |
| | Chloride, | cyclopentadienyl)- | ruthenium(II), | (III) Chloride, |
| Catalyst | Dimer | dirhodium | Dimer | Dimer |
| S,S-N-2,4,6-tri- | Racemic | Racemic | Racemic | Racemic |
| iPr-Bs-DACH | | | | |
| S,S-N-F5Bs- | 91% | 94% | 96.3% | 80% |
| DPEN | | | | |
| S,S-N-Ts- | 93% | 96% | 96% | 74% |
| DPEN | | | | |
| S,S-N-Ts- | 92% | 87% | 95% | 67% |
| DACH | | | | |
| S,S-N- | 92% | 90% | 95.6% | 73% |
| naftalene-2- | | | | |
| sulfonyl-DACH | | | | |
| S,S-N-Me5Bs- | 92% | Racemic | 96.5% | racemic |
| DACH | | | | |
| (1R,2S)-cis-1- | Racemic | Racemic | Racemic | Racemic |
| amino-2- | | | | |
| indanol | | | | |
| S,S-N- | 91% | 95.6% | 96% | 70% |
| naphtalene-2- | | | | |
| sulfonyl-DPEN | | | | |
| S,S-N-3,5-di- | 87% | 96% | 94% | 90% |
| CF3-Bs-DACH | | | | |
| S,S-N-Me5Bs- | 89% | 60% | 96.9% | 64% |
| DPEN | | | | |
| S,S-N-2,4,6-tri- | 88% | No reaction | 96% | 64% |
| iPr-Bs-DPEN | | | | |
| S,S-N- | 92% | 97% | 95% | 93% |
| methanesulfonyl- | | | | |
| DPEN | | | | |

1. A process for the preparation of the compound 1-[5-(3-chloro-phenyl)-isoxazol-3-yl]-ethanone: 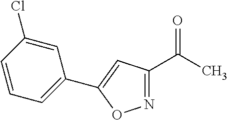
wherein
the compound ethyl 5-(3-chlorophenyl)-isoxazole-3-carboxylate of the formula 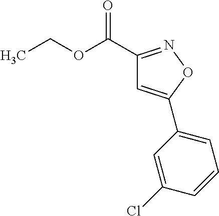
dissolved in a solvent, is reacted with CH₃MgX dissolved in a solvent, wherein X is chlorine or bromine, thereby providing the compound 1-[5-(3-chloro-phenyl)-isoxazol-3-yl]-ethanone dissolved in said solvent.

2. A process according to claim 1, wherein the solvent is toluene, or ortho-, meta-, or para-xylene.

3. A process according to claim 1, wherein the solvent is an ether selected from 2-methyl tetrahydrofuran, tetrahydrofuran, diethyl ether, tert-butyl methyl ether or a mixture thereof.

4. A process according to claim 1, wherein the solvent is a mixture of one or more of toluene, ortho-, meta-, or para-xylene, 2-methyl tetrahydrofuran, tetrahydrofuran, diethyl ether, tert-butyl methyl ether

5. A process according to claim 1, wherein the reaction is carried out in the presence of a tertiary amine.

6. A process according to claim 5, wherein the tertiary amine is triethyl amine, tri-N-butylamine or an N-alkylpiperidine.

7. A process according to claim 1, wherein the reaction mixture and surplus of said CH₃MgX is quenched by (i) adding an acid aqueous solution followed by (ii) addition of a basic aqueous solution.

8. A process according to claim 7, wherein the acid aqueous solution is HCl.

9. A process according to claim 8, wherein the HCl is 6 M HCl.

10. A process according to claim 7, wherein the basic aqueous solution is sodium hydroxide.

11. A process according to claim 1, wherein 1-[5-(3-chloro-phenyl)-isoxazol-3-yl]-ethanone is isolated by crystallization from a mixture of 2-methyl tetrahydrofuran and n-heptene.

12. A process for preparing (R)-1-[5-(3-chloro-phenyl)isoxazol-3-yl]-ethanol of the formula 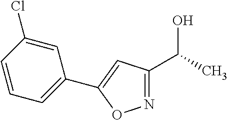
comprising the steps of:
i) preparing 1-[5-(3-chloro-phenyl)-isoxazol-3-yl]-ethanone;
ii) providing (S)-2-methyl-CBS oxaborolidine and borane or a borane complex dissolved in a first solvent;
iii) adding 1-[5-(3-chloro-phenyl)-isoxazol-3-yl]-ethanone dissolved in a solvent to the solution obtained in step ii); and
iv) recovering (R)-1-[5-(3-chloro-phenyl)-isoxazol-3-yl]-ethanol.

13. A process according to claim 12, wherein (S)-2-methyl-CBS oxaborolidine and a borane complex, is dissolved in the first solvent of step ii, and in that said first solvent is selected from tetrahydrofuran or 2-methyl tetrahydrofuran.

14. A process according to claim 12, wherein the borane complex is selected from any one of borane dimethyl sulfide, borane tetrahydrofuran, borane triethylamine and borane N,N-diethylaniline.

15. A process according to claim 12, wherein an excess of borane is quenched by adding an alcohol, after completion of formation of (R)-1-[5-(3-chloro-phenyl)-isoxazol-3-yl]-ethanol.

16. A process according to claim 15, wherein the alcohol is methanol.

17. A process according to claim 15, wherein (R)-1-[5-(3-chloro-phenyl)-isoxazol-3-yl]-ethanol is recovered by crystallization from a single solvent, or a mixture of solvents.

18. A process according to claim 17, wherein the single solvent is toluene or xylene.

19. A process according to claim 17, wherein the solvent is a mixture of toluene and n-heptane.

20. A process according to claim 17, wherein the solvent is 2-methyl tetrahydrofuran, tetrahydrofuran, diethyl ether or tert-butyl methyl ether in combination with a second solvent.

21. A process according to claim 20, wherein the second solvent is an alkane.

22. A process according to claim 21, wherein the second solvent is n-heptane.

23. A process according to claim 17, wherein the solvent is an apolar aprotic solvent in combination with second solvent.

24. A process according to claim 23, wherein the apolar aprotic solvent is dimethylsulfoxide, or dimethylformamide.

25. A process according to claim 23, wherein the second solvent is water.
